(19) United States
(12) Patent Application Publication
(10) Pub. No.: US 2026/0165753 A1
(43) Pub. Date: Jun. 18, 2026

(54) Endoprosthesis System with Cortical Clamping

(71) Applicant: Howmedica Osteonics Corp., Mahwah, NJ (US)

(72) Inventors: Brad Parker, Warsaw, IN (US); Anthony Andreas, Columbia City, IN (US); Doug Garman, Fort Wayne, IN (US); Matt Kartholl, Fort Wayne, IN (US); Travis Geels, Fort Wayne, IN (US)

(73) Assignee: Howmedica Osteonics Corp., Mahwah, NJ (US)

(21) Appl. No.: 19/416,391

(22) Filed: Dec. 11, 2025

Related U.S. Application Data
(60) Provisional application No. 63/734,229, filed on Dec. 16, 2024.

(51) Int. Cl. A61B 17/72; A61F 2/30; A61F 2/46

(52) U.S. Cl. CPC A61B 17/7233; A61F 2/30734; A61F 2/30744; A61F 2002/30729; A61F 2002/3085; A61F 2002/4622

(57) ABSTRACT

A bone clamp for coupling an intramedullary stem to a bone includes a collet, a bushing, and a cap. The collet includes a main body and a plurality of legs and is configured to receive the bone and the intramedullary stem. The collar includes internal threading that is complementary to external threading on the main body of the collet such that rotation of the collar relative to the collet translates the collar to compress the plurality of legs radially inwardly against the bone. The bushing is received within the collet, and the cap includes internal threading that is complementary to external threading on the proximal end of the collet such that rotation of the cap relative to the collet translates the cap distally relative to the collet to compress the bushing against the intramedullary stem.

### CROSS REFERENCE TO RELATED APPLICATIONS

This application claims the benefit of the filing date of U.S. Provisional Application No. 63/734,229, filed Dec. 16, 2024, the disclosure of which is hereby incorporated by reference herein in its entirety.

### BACKGROUND

Over time, repeated use of bones and joints can lead to damage or wear. Factors such as repetitive strain from athletic activities, traumatic injuries, and diseases like arthritis can cause the cartilage, which cushions joints, to deteriorate. As the cartilage deteriorates, fluid can accumulate in the joints, leading to pain, stiffness, and reduced mobility. Similar issues can arise when tendons become lax or when soft tissues surrounding the joint are damaged or worn.

Arthroplasty is a surgical procedure used to repair damaged joints. During arthroplasty, an arthritic or dysfunctional joint is reshaped or realigned, often with the insertion of one or more prosthetic implants. This type of procedure can be performed on various joints in the body, including the knees, hips, shoulders, and elbows. In shoulder arthroplasty, for example, a damaged shoulder joint is replaced with prosthetic implants, typically due to conditions such as severe osteoarthritis, trauma, or joint disease.

In more severe cases, skeletal defects from trauma or bone tumors may necessitate the complete removal of an affected bone. This resection usually occurs along the diaphysis of a long bone, such as the femur, tibia, or humerus. In such instances, a portion of the diaphysis is removed along with the entirety of the metaphysis, epiphysis, and articular structure of the bone. An endoprosthesis, also known as a megaprosthesis, may then be implanted by inserting an intramedullary stem into the remaining diaphysis, replacing both the joint and the bone structure that has been removed. This type of arthroplasty procedure is sometimes referred to as a limb salvage procedure.

### BRIEF SUMMARY OF THE DISCLOSURE

The present disclosure describes various devices, systems, and methods for securing an endoprosthesis to a bone, such as a diaphysis of the bone. Such devices and systems may include an endoprosthesis with a stem that may help secure the endoprosthesis from within the bone, and a bone clamp (or cortical engagement assembly) which may help secure the endoprosthesis to a cortical shell at the exterior of the bone allowing for load sharing and resistance to moments, which may reduce the risk of periprosthetic fractures.

In one aspect of the present disclosure, a bone clamp for coupling an intramedullary stem to a bone in which the intramedullary stem is received includes a collet, a bushing, a plurality of legs, and a cap. The collet includes a main body, and the plurality of legs extend distally from the main body. The collet forms an internal channel which is configured to receive at least a portion of the bone and at least a portion of the intramedullary stem therethrough.

The collar includes internal threading that is complementary to external threading on the main body of the collet. The collar is configured to be received around the main body of the collet such that rotation of the collar relative to the collet translates the collar distally relative to the collet to compress the plurality of legs radially inwardly.

The bushing is configured to be received within the proximal end of the collet. The bushing has a longitudinal channel configured to receive at least a portion of the intramedullary stem therethrough. The bushing is generally annular with a gap formed between two walls of the bushing that confront each other.

The cap has internal threading that is complementary to external threading on the proximal end of the collet. The cap has a longitudinal channel configured to receive at least a portion of the intramedullary stem therethrough. The cap also has a distal face configured to contact a proximal face of the bushing. The cap is configured to be received around the proximal end of the collet such that rotation of the cap relative to the collet translates the cap distally relative to the collet to compress the bushing and to reduce the gap of the bushing and compress the bushing onto the intramedullary stem.

Additionally, the legs of the collet may be integrally formed with the main body so as to form a monolithic structure. The legs may be cantilevered to the main body and may be deflectable from a first position to a second position. The legs may be biased towards the first position. The legs may each have an exterior surface, and the collar may include a cam surface engaging the exterior surface of each of the legs such that driving the collar in a distal direction cams the legs towards the second position. In one example, the legs include three legs. The collar may include tool engagement features on an outer surface thereof for engagement with a torque applying tool for rotating the collar and driving the collar in the distal direction.

Also, the main body may include a proximal end portion that may define the proximal end portion of the collet. The proximal end portion may have the external threading which is complementary to the internal threading of the cap. The proximal end portion of the main body may include a tapered inner surface, and the bushing may be received within the inner surface and may include a tapered outer surface complementary to the tapered inner surface of the proximal end portion such that driving the bushing in a distal direction closes the gap between the two walls of the bushing. The main body may also include a distal end portion that may define a distal end of the collet. The distal end portion may have the external threading which is complementary to the internal threading of the collar.

Furthermore, the main body may include a circumferential shoulder at an interior thereof for receipt of a corresponding flange of the intramedullary stem. The circumferential shoulder may be disposed distally to the bushing such that driving the bushing distally when the flange of the intramedullary stem abuts the circumferential shoulder causes the bushing to engage the flange. In some examples, the bushing is eccentric such that a longitudinal axis of the bushing is offset relative to a longitudinal axis of the collet. The bushing may be made from a compliant material. Also, the bushing may include a circumferential flange disposed between the proximal end of the collet and the cap so as to constrain distal movement of the bushing.

In another aspect of the present disclosure, a bone clamp for coupling an intramedullary stem to a bone in which the intramedullary stem is received includes a collet, a bushing, a plurality of legs, and a cap. The collet includes a main body that defines a proximal end portion which itself defines a proximal end of the collet. The main body forms an internal channel configured to receive at least a portion of the bone and at least a portion of the intramedullary stem therethrough. The main body has a plurality of openings formed in a side wall thereof. The openings are spaced apart from each other in a circumferential direction of the main body

Each of the plurality of legs have a distal portion with an inner surface configured to contact the bone. The legs also have a proximal portion each with a protrusion. Each protrusion is configured to be received within any one of the openings formed in the side wall of the main body to couple the corresponding leg to the main body.

The bushing is configured to be received within the proximal end portion of the main body. The bushing has a longitudinal channel configured to receive at least a portion of the intramedullary stem therethrough. The bushing is generally annular with a gap formed between two walls of the bushing that confront each other.

The cap has internal threading that is complementary to external threading on the proximal end portion of the main body. The cap has a longitudinal channel configured to receive at least a portion of the intramedullary stem therethrough. The cap has a distal face configured to contact a proximal face of the bushing. Also, the cap is configured to be received around the proximal end portion of the main body such that rotation of the cap relative to the main body translates the cap distally relative to the main body to compress the bushing and to reduce a size of the gap of the bushing.

Additionally, the inner surface of each of the plurality of legs may include a porous structure. Further, the inner surface of each of the plurality of legs may be flat. The proximal portion of each of the legs may define a first longitudinal axis, and the distal portion of each of the legs may define a second longitudinal axis. The legs may include a first leg and a second leg.

In some implementations of the first leg, the first longitudinal axis of the proximal portion is offset relative to the second longitudinal axis of the distal portion of the first leg. In one example, the second longitudinal axis of the distal portion of the first leg is offset radially inwardly relative to the first longitudinal axis of the proximal portion when the first leg is connected to the main body. In another example, the second longitudinal axis is offset radially outwardly relative to the first longitudinal axis of the proximal portion when the first leg is connected to the main body. In other implementations of the first leg, the first longitudinal axis is coaxial with the second longitudinal axis.

In some implementations of the second leg, the first longitudinal axis of the proximal portion is offset relative to the second longitudinal axis of the distal portion of the second leg. In one example, the second longitudinal axis of the distal portion of the second leg is offset radially inwardly relative to the first longitudinal axis of the proximal portion when the second leg is connected to the main body. In another example, the second longitudinal axis is offset radially outwardly relative to the first longitudinal axis of the proximal portion when the second leg is connected to the main body. In other implementations of the second leg, the first longitudinal axis is coaxial with the second longitudinal axis.

In a further aspect of the present disclosure, method of securing an intramedullary stem to a bone in which the intramedullary stem is received includes positioning a main body of a bone clamp onto a resected end of the bone so that a bushing received within a proximal end of the main body is received around a portion of the intramedullary stem. The method also includes positioning a plurality of legs of the bone clamp, which extend distally from the main body, around a portion of the bone so that inner faces of the plurality of legs are in contact with an outer cortical surface of the bone. The method further includes contracting the bushing onto the portion of the intramedullary stem to secure the intramedullary stem while the plurality of legs are in contact with the outer cortical surface of the bone, and while the intramedullary stem is received within the bushing,

Additionally, the contracting step may include driving the bushing in a first direction relative to the main body such that a tapered inner surface of the main body interacts with a tapered outer surface of the bushing to cam the bushing radially inwardly. The bushing may include includes two walls which may define a gap therebetween. Also, the driving step may include moving the two walls toward each other. The bone clamp may also include a cap threadedly engaged to the main body, and the driving step may include rotating the cap such that an engagement surface of the cap engages the bushing and drives the bushing in the first direction.

Furthermore, the method may include moving the plurality of legs radially inwardly into engagement with the bone. The moving step may include moving a collar disposed over the main body in a first direction such that a cam surface of the collar engages the plurality of legs and moves the plurality of legs radially inwardly. The collar may be threadedly engaged to the main body, and the step of moving the collar in the first direction may include rotating the collar in a first rotational direction.

Also, the method may further include measuring an offset between the outer cortical surface of the bone relative to an outer surface of the main body. Based on the measuring step, the method may include selecting a first leg of the plurality of legs corresponding to the measured offset. Further, the method may include connecting the first leg to the main body of the bone clamp. The measuring step may include inserting a trial stem into the bone and positioning a main body sizer over an end of the trial stem. The main body sizer may have a size corresponding to a size of the main body. The measuring step may also include placing a sizing instrument against an outer surface of the main body sizer and against the outer cortical surface of the bone. The main body sizer may include a plurality of flat surfaces arrayed about a longitudinal axis of the main body sizer. The sizing instrument may also include a plurality of stepped surfaces each corresponding to a bone offset. The measuring step may also include placing a first stepped surface against the outer cortical bone surface and a second stepped surface against one of the flat surfaces of the main body sizer.

### BRIEF DESCRIPTION OF THE DRAWINGS

FIG. 1 is an elevational view of an endoprosthesis system according to an embodiment of the present disclosure.

FIG. 2 is an elevational view of an exemplary endoprosthesis of the endoprosthesis system of FIG. 1.

FIG. 3A is an elevational view of an exemplary bone clamp of the endoprosthesis system of FIG. 1.

FIG. 3B is a cross-sectional view of the bone clamp of FIG. 3A taken along a midline thereof.

FIG. 4A is a perspective view of an exemplary mounting body of the bone clamp of FIG. 3A.

FIG. 4B is a perspective view of an exemplary kit of differently sized mounting bodies including the mounting body of FIG. 4A.

FIG. 5A is a perspective view of an exemplary cortical engagement member of the bone clamp of FIG. 3A.

FIG. 5B is an elevational view of an exemplary kit of differently configured cortical engagement members including the cortical engagement member of FIG. 5A.

FIG. 6A is an elevational view of a partial assembly of the bone clamp of FIG. 3A in a first configuration.

FIG. 6B is a schematic cross-sectional view of the partial assembly of FIG. 6A in association with a bone.

FIG. 7A is an elevational view of a partial assembly of the bone clamp of FIG. 3A in a second configuration.

FIG. 7B is a schematic cross-sectional view of the partial assembly of FIG. 7A in association with a bone.

FIGS. 8-12 illustrate a method of implanting the endoprosthesis system of FIG. 1.

FIG. 13A is a perspective view of a bone clamp according to another embodiment of the present disclosure.

FIG. 13B is a cross-sectional view of the bone clamp of FIG. 13A taken along a midline thereof.

FIG. 13C is an exploded view of the bone clamp of FIG. 13A and relative to a bone.

FIG. 15A is a perspective view of a bone clamp according to a further embodiment of the present disclosure.

FIG. 15B is a perspective view of an exemplary kit of differently sized cortical engagement assemblies including the bone clamp of FIG. 15A.

FIG. 16 is a perspective view of a bone clamp according to another embodiment of the present disclosure.

FIG. 17 is a perspective view of a mounting body of the bone clamp of FIG. 16 according to an alternative embodiment of the present disclosure.

### DETAILED DESCRIPTION

As used herein, the term “proximal” refers to the end of a surgical tool or device closer to the user during its intended use, while “distal” refers to the end farther from the user. When referring to the human body, “proximal” means closer to the heart, and “distal” means farther from the heart. The terms “substantially,” “generally,” “approximately,” and “about” are used to include slight deviations from the absolute, typically up to 10% more or less. All vertical directional terms, such as “up,” “down,” “above,” “below,” “vertical,” or “height,” refer to the orientation shown in the figures and are not meant to suggest any specific orientation for the device when constructed.

In limb salvage procedures, where a significant portion of a long bone may be removed, an intramedullary stem is often inserted into the remaining bone to secure the endoprosthesis within the intramedullary canal. One drawback of this type of fixation, especially for endoprostheses of this kind, is the generation of substantial bending moments at the bone-prosthesis interface. As a result, the risk of periprosthetic fractures in these procedures is relatively high as compared to arthroplasties that only replace the bone's articular surface.

To mitigate this risk, it may be desirable to share the load by also securing the prosthesis to the bone's exterior. However, this is complicated by the fact that the exterior geometry of bones, particularly the diaphysis of long bones, is neither uniform nor perfectly cylindrical. Instead, the diaphysis often has a non-uniform shape with various protuberances, which can vary from patient to patient. The following describes exemplary devices, systems, and methods for securing an endoprosthesis internally and externally to a bone.

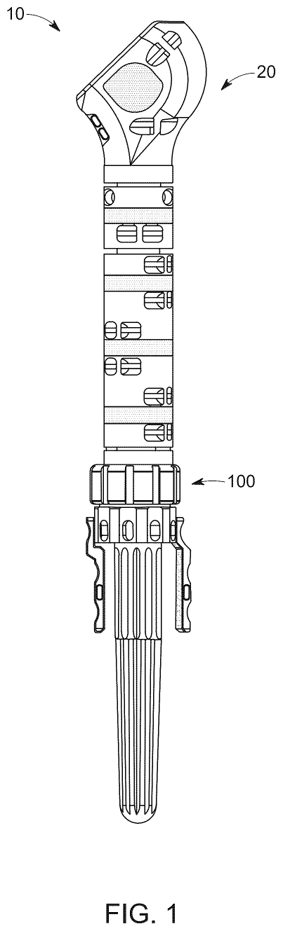

FIGS. 1-7B depict an endoprosthesis system 10 according to an embodiment of the present disclosure. Endoprosthesis system 10 generally includes an endoprosthesis 20 and a bone clamp 100.

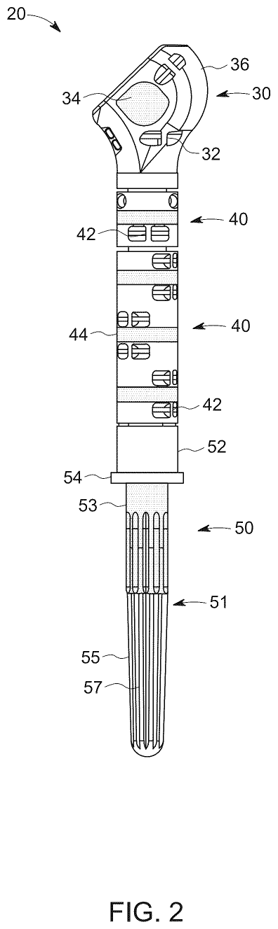

FIG. 2 depicts an exemplary endoprosthesis 20 that may be utilized in system 10. In the embodiment depicted, endoprosthesis 20 is constructed for replacement of a proximal humerus, such as in a limb salvage procedure. Although, endoprosthesis 20 is configured to replace a proximal humerus, it should be understood that endoprosthesis 20 can be any endoprosthesis configured to replace any portion of any of the long bones of a mammalian subject. Endoprosthesis 20 generally includes a proximal body 30, one or more spacers 40, and a stem component 50.

Proximal body 30 (or metaphyseal body) is configured to replicate a proximal end of a humerus and, as such, may have a plurality of curved and/or flat surfaces that may mimic the structure of a native proximal humerus. Proximal body 30 may also have an articular surface connected thereto. Proximal body 30 may include a plurality of eyelets 32 and/or porous portions 34 for the connection of native soft tissues thereto. The geometry of proximal body 30 is configured to atraumatically interact with the soft tissues during normal articulations so as to not abrade or otherwise damage the soft tissues and so that the native soft tissues are operable to provide joint articulation. For example, proximal body 30 may include a spherical surface 36 at a lateral side thereof to promote deltoid wrapping.

One or more spacers 40 may be connected to proximal body 30 to build up a desired length of endoprosthesis 20 to accommodate a length of the bone that was removed. In this regard, spacers 40 are configured mimic the diaphyseal portion of the resected bone. Additionally, spacers 40 are configured to be modularly connected to each other, proximal body 30, and stem component 50 to obtain the desired length. The connection between spacers 40, proximal body 30, and/or stem component 50 may be via a Morse taper or the like. Spacers 40 may also include a plurality of eyelets 42 and/or porous portions 44 for soft tissue connection thereto. For example, eyelets 42 may be configured to receive sutures or wires (e.g., cerclage wires) to secure soft tissue thereto, and spacers 40 may include porous portions 44 adjacent to such eyelets 42 to facilitate tissue ingrowth or ongrowth.

Stem component 50 (or intramedullary stem), as shown, includes an adapter 52 and a stem 51 extending from adapter 52. Adapter 52 may be integral with stem 51 so as to form a monolithic structure, or alternatively may be modularly connected to stem 51, such as via a Morse taper or the like. As mentioned above, spacers 40 may be connected to adapter 52, which may be via a Morse taper or the like. Similarly, proximal body 30 may be directly connected to adapter 52 in circumstances where spacers 40 are not needed. Adapter 52 may be cylindrical and may include a flange 54 extending radially outwardly therefrom. In some embodiments, adapter 52 may include indentations or other features (not shown), such as snap-fit features, for connection to trial components, such as trial spacers, for example. As shown, flange 54 may be annular, and stem 51 may extend distally from flange 54. Stem 51 may be conical or conical and cylindrical, for example. Stem 51 may include a proximal portion 53 and a distal portion 55. Proximal portion 53 may include a porous exterior for bone ingrowth or ongrowth. For example, proximal portion 53 may have a titanium plasma spray coating. Distal portion 55 may not include a porous structure and instead may have a solid non-porous exterior. Stem 51 may also include a plurality of flutes or splines 57 to help constrain rotation within an intramedullary canal. Exemplary endoprostheses including exemplary proximal bodies, spacers, and stem components, which may be utilized in endoprosthesis system 10, are further described in U.S. Publication No. 2023/0310167 and U.S. application Ser. No. 18/893,096, the disclosures of which are incorporated by reference herein in their entireties.

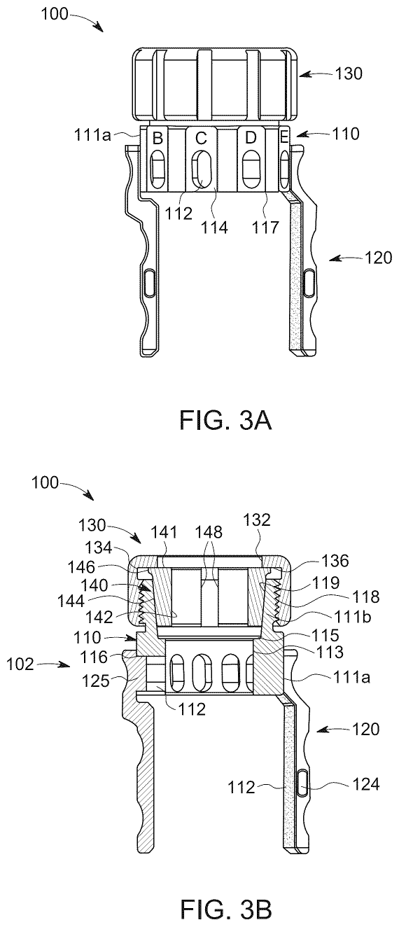

FIGS. 3A-7B depict bone clamp 100 according to an embodiment of the present disclosure. Bone clamp 100 (or cortical engagement assembly) generally includes a collet 102, a cap 130, and a bushing 140.

Collet 102 generally includes a main body 110 and a plurality of legs 120. In the embodiment depicted, legs 120 are modular in that they can be interchangeably connected to main body 110. Additionally, collet 102 is a fixed-leg collet in that legs 120, once connected to main body 110, are stationary and do not move inwardly to engage the bone as their modular construction allows for the selection of legs 120 that securely engage the bone in a fixed state, as described in more detail below. However, in some embodiments of collet 102, legs 120 may be flexible and, once connected to main body 110, may be cantilevered thereto so as to flex radially inwardly and outwardly.

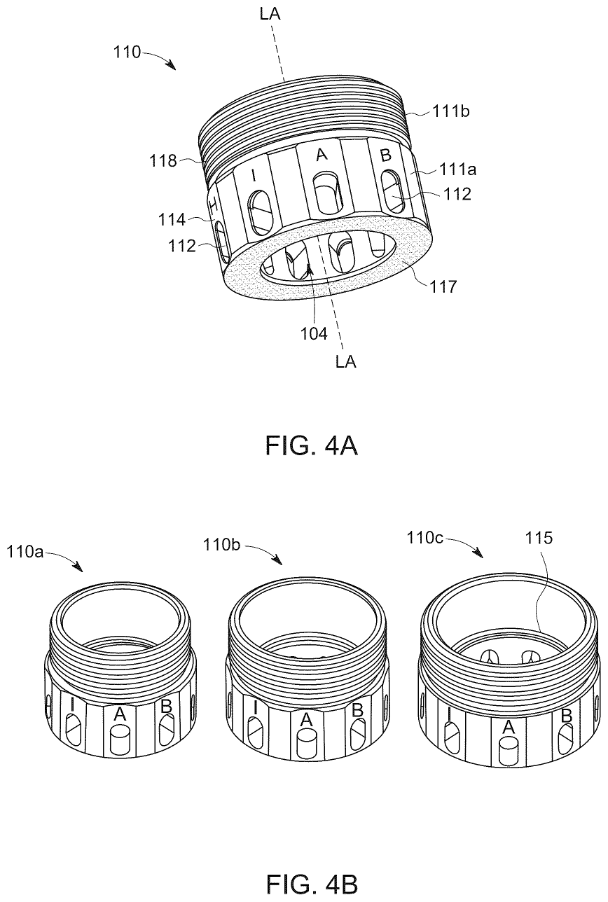

FIG. 4A depicts main body 110. Main body 110 (or baseplate) may have a cylindrical shape exterior shape and cylindrical interior shape and has a longitudinal channel 104 extending therethrough which defines a longitudinal axis LA of main body 110 and of bone clamp 100. However, in some embodiments, the exterior shape of main body 110 may differ, for example, the exterior shape of main body 110 may be rectangular, hexagonal, octagonal, and the like. Additionally, in some embodiments, the interior shape of main body 110 may be conical to match a conical taper of a stem 51, for example. Main body 110 includes a first portion 111a (or distal portion) and a second portion 111b (or proximal portion). Distal portion 111a includes a distal surface 117 which is configured to engage a resected surface of a bone when implanted. For example, a diaphysis of bone may be cut so as to expose a planar resected surface. Distal surface 117 may be correspondingly planar so as lay flush against the resected surface when implanted. Distal surface 117 may also have a porous structure to facilitate bone ongrowth or ingrowth. For example, main body 110 may be made via an additive manufacturing processes which may form a porous structure at its distal end. In other examples, distal surface 117 may be coated with a porous material, such as a titanium plasma spray, for example.

Distal portion 111a of main body 110 may include a plurality of facets 114 (or flat surfaces) on an exterior of main body 110. As shown, facets 114 are arrayed about the longitudinal axis LA of main body 110, and each include an opening 112 (or slot) extending therein. Such openings 112 may extend entirely through main body 110 in a radial direction so as to intersect channel 104. However, in some embodiments, openings 112 may be blind openings such that they do not extend into channel 104. Openings 112 may be oval or pill-shaped, as shown. However, in other embodiments, openings 112 may be circular, rectangular, or the like. As shown in FIG. 3B, each opening 112 may also include a lip 116 (see FIG. 3B) which may be configured to correspondingly engage with a lip of a connection feature of a leg 120, as described further below. Each facet 114 and corresponding opening 112 may have a unique identifier which indicates the opening's position about the perimeter of distal portion 111a. Such identifier may be etched or otherwise located on facet 114 and may be used to identify an appropriate location for connected of a leg 120 selected during a trialing procedure, as described further detail. Such identifier may be a letter, a number, or the like, for example.

Proximal portion 111b of main body 110 defines a proximal end of collet 102. As shown, proximal portion 111b includes external threading 118 for corresponding threaded engagement with cap 130. However, in other embodiments, proximal portion 111b may have alternative features for connecting to cap 130, such as teeth of a ratchet mechanism, for example. Proximal portion 111b also includes an inner surface 119 which may be a tapered surface that tapers inwardly in a distal direction toward distal portion 111a. An annular shoulder 115 (or rim) may be formed on an interior of main body 110 and may be formed at a junction between proximal portion 111b and distal portion 111a. Such annular shoulder 115 is configured to receive flange 54 of stem component 50 such that flange 54 may abut shoulder 115 when stem component 50 is received within channel 104 of main body 110.

FIG. 14B depicts an exemplary kit of main bodies like that of main body 110. Such kit may include a first main body 110a, a second main body 110b, and a third main body 110c each of different size. For example, first main body 110a may have a nominal diameter size of 27 mm, second main body 110 b may have a nominal diameter size of 24 mm, and third main body 110 c may have a nominal diameter size of 21 mm. Additionally, kit can accommodate stems of various nominal diameters, such as 9 mm to 19 mm, for example. Such a kit of different sized main bodies 110a-c allows a surgeon to select an appropriately sized main body 110 to correspondingly match the cross-sectional dimension of a patient's bone.

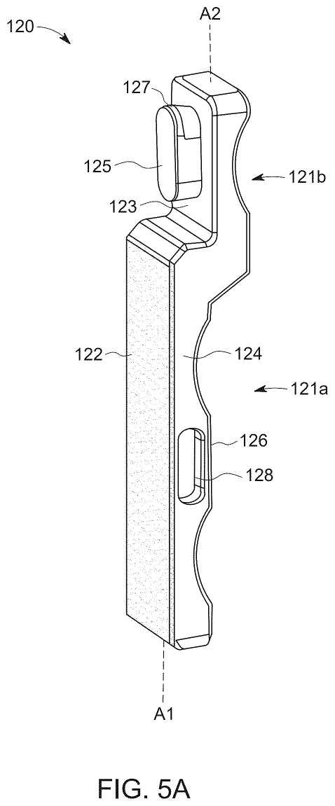

FIG. 5A depicts an exemplary leg 120 of bone clamp 100. Leg 120 generally includes a first portion 121a (or distal portion) and a second portion 121b (or proximal portion). Distal portion 121a has an axial length that defines a longitudinal axis A1. Distal portion 121a includes an inner surface 122, an outer surface 126 disposed opposite inner surface 122, and a sidewall 124 extending therebetween. Inner surface 122 (or bone engagement surface) is configured to engage cortical bone. In the embodiment depicted, inner surface 122 is flat and includes a porous structure to promote bone ingrowth or ongrowth. However, in some embodiments, inner surface 122 may not be porous and may instead be smooth, roughened, or have corrugations, for example. In yet further embodiments, inner surface 122 may have spikes, such as conical spikes, configured biting into the hard cortical shell. Inner surface 122 may also alternatively be curved, such as concavely curved about the longitudinal axis LA of main body 110, for example. As shown, each sidewall 124 extending between inner and outer surfaces 122, 126 may have a tool engagement feature 128 for engaging a grasping tool, as described further below. Such engagement feature 128 may be an indentation or may be a projection, for example.

Proximal portion 121b of leg 120 extends proximally from distal portion 121a and has an axial length that defines a longitudinal axis A2. Longitudinal axis A1 of distal portion 121a may be coaxial with longitudinal axis A2 of proximal portion 121b or may be offset relative to longitudinal axis A2 of proximal portion 121b, as described further below. In embodiments in which axis A1 and axis A2 are offset, it is preferable that such axes A1, A2 are parallel to each other. However, it is contemplated that axes A1 and A2 may be angled relative to each other. Proximal portion 121b has an inner surface 123 and a connection feature 125 extending from inner surface 123. Connection feature 125 is protrusion configured to be received within any one of openings 112 of main body 110. As such, connection feature 125 may be similarly shaped to that of openings 112, which in the embodiment depicted, is ovular or pill shaped. Connection feature 125 may also have a lip 127 (or flange) extending proximally therefrom. Such lip 127 is configured to engage the corresponding lip 116 within each opening 112 of main body 110 so as to secure leg 120 to a selected opening 112 and prevent leg 120 from being inadvertently removed from opening 112. However, it should be understood that other connection mechanisms may be implemented, such as a snap-fit mechanism, press-fit mechanism, or threaded fastener mechanism, for example. Additionally, while main body 110 is shown as having an opening 112 (or female part) and leg 120 is shown as having a protrusion 125 (male part) for their connection, main body 110 may alternatively have the male part, while leg may have the female part. Inner surface 123 of proximal portion 121b of leg 120 may be flat such that when connection portion 125 is received within main body 110, inner surface 123 lays flush against the corresponding facet 114. Inner surface 123 of proximal portion 121b may be parallel to or coplanar with inner surface 122 of distal portion 121a.

FIG. 5B depicts an exemplary kit of legs like that of leg 120. Each leg of the kit may have a different offset to accommodate variations in overlap between main body 110 and a bone. In particular, when main body 110 is placed onto a resected end of a bone with channel 104 aligned with an intramedullary canal of the bone, main body 110 may overlap the cortical outer surface of the bone such that main body 110 projects radially outwardly relative to the bone. Conversely, the outer cortical surface of the bone may project radially outwardly further than main body 110, or main body 110 may be flush with the outer cortical surface. Furthermore, some or all of these conditions may exist at various locations about the longitudinal axis LA of main body 110. Legs 120 with varying offset configurations may be connected to main body 110 to accommodate each of these conditions to ensure a snug fit against the cortical shell for load sharing and moment resistance.

Thus, in the example kit a plurality of legs 120a-g may be provided each with varying degrees of offset, such as an inward offset, a flush offset, and outward offset. For example, a first leg 120a, a second leg 120b, a third leg 120c, and a fourth leg 120d may each have an inward offset such that distal portion 121a of each of these legs 120a-d is offset inwardly relative to proximal portion 121b. In other words, when legs 120a-d are connected to main body 110, inner surface 122 of distal portion 121a and axis A1 of distal portion 121a is positioned closer to longitudinal axis LA of main body 110 than inner surface 123 of proximal portion 121b and axis A2 of proximal portion 121b. As illustrated, the inward offset (or negative offset) of first leg 120a is shown as X1, the inward offset of second leg 120b is shown as X2, the inward offset of third leg 120c is shown as X3, and the inward offset of fourth leg 120d is shown as X4. X1 is greater than X2, which is greater than X3, which is greater than X4. Such offsets X1-X4 may be in consistent increments, such as 1 mm increments, for example. Thus, in the embodiment shown, first offset X1 may be 4 mm, second offset X2 may be 3 mm, third offset X3 may be 2 mm, and fourth offset X4 may be 1 mm, for example. In other embodiments, the incremental difference may be 0.5 mm for example, and more than four inward-offset legs 120 may be provided to provide a full range of possibilities. Any one of these legs 120a-d may be used in locations in which the outer cortical surface of the bone is inwardly offset (or inset) relative to an outer surface or facet 114 of main body 110.

Additionally, a fifth leg 120e of the kit may have a neutral offset. In this regard, the inner surface 123 of proximal portion 121b and inner surface 122 of distal portion 121a are flush, and longitudinal axis A1 and A2 are coaxial. Such leg 120e may be used in locations where the outer cortical surface of bone is flush with an outer surface or facet 114 of main body 110.

Further, a sixth leg 120f and a seventh leg 120g may each have an outward offset such that distal portion 121a of each of these legs 120f-g is offset outwardly relative to proximal portion 121b. In other words, when legs 120f-g are connected to main body 110, inner surface 122 of distal portion 121a and axis A1 of distal portion 121a is positioned further from the longitudinal axis LA of main body 110 than inner surface 123 of proximal portion 121b and axis A2 of proximal portion 121b. As illustrated, the outward offset (or positive offset) of sixth leg 120f is shown as X6, and the outward offset of seventh leg 120g is shown as X7. X7 is greater than X6. Such offsets X6, X7 may have the same increments as the inward-offset legs 120a-d, such as 1 mm increments, for example. Thus, in the embodiment shown, sixth offset X6 may be 1 mm, and seventh offset X7 may be 2 mm, for example. Any one of these legs 120f-g may be used in locations in which the outer cortical surface of the bone is outwardly offset (or outset) relative to an outer surface or facet 114 of main body 110.

Although seven legs 120a-g are depicted in the kit of FIG. 5B, more or less legs 120 may be provided to account for variations in bone geometries and number of different sized main bodies 110 that may be provided. For example, more or less inward-offset legs 120 may be provided, and/or more or less outward-offset legs 120 may be provided. Also, as shown in FIG. 5B, the distal portion 121a of each leg 120a-g has a length L which may be the same amongst each leg 120a-g in the kit. However, in other embodiments, length L may differ between each leg 120a-g or multiple groups of legs 120a-g may be provided with each group having a different length L and having all of the different offsets described above.

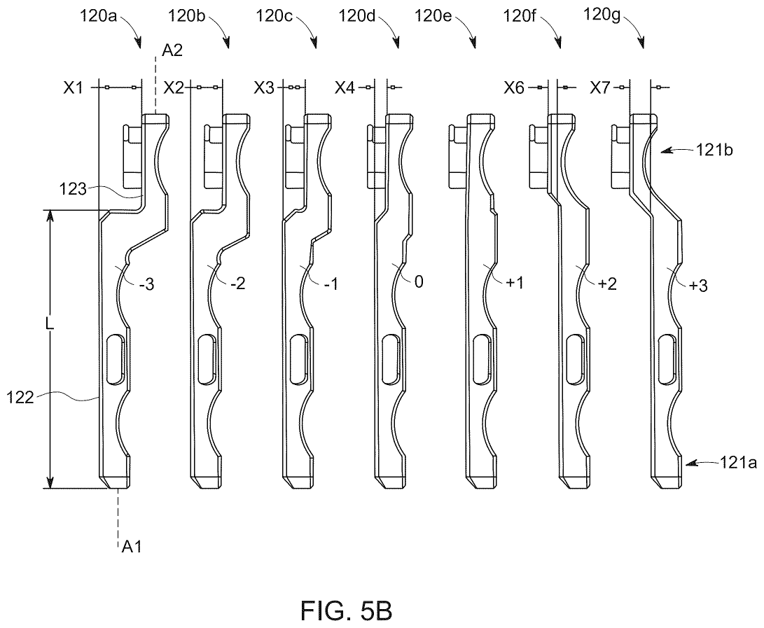

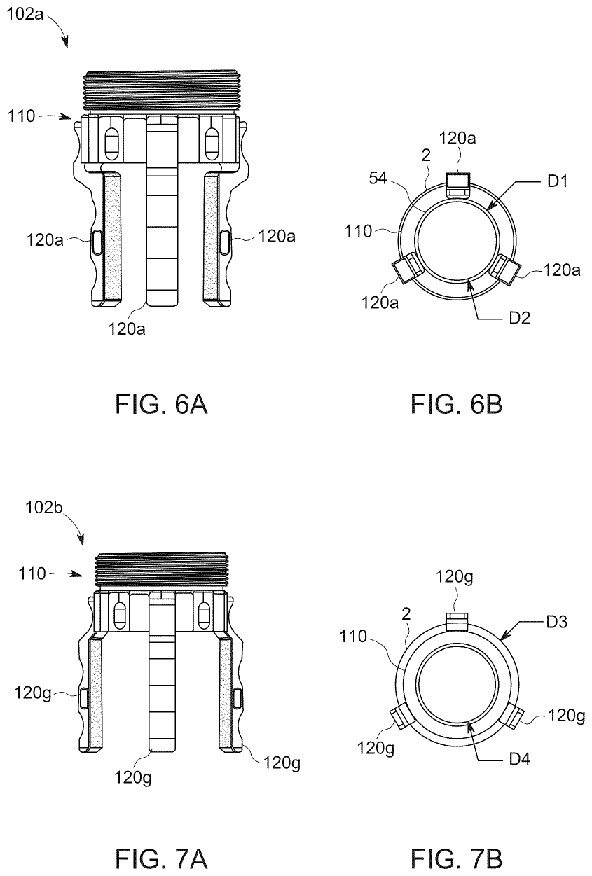

FIGS. 6A and 6B illustrate the narrowest configuration of collet 102. In this configuration, each leg connected to main body 110 is a leg with the greatest inward offset, such as first leg 120a. Depending on the size of main body 110, this configuration may accommodate humeral diameters from 17 mm to 22 mm.

FIGS. 7A and 7B illustrate the widest configuration of collet 102. In this configuration, each leg connected to main body 110 is a leg with the greatest outward offset, such as seventh leg 120a. Depending on the size of main body 110, this configuration may accommodate humeral diameters from 29 mm to 34 mm. Thus, endoprosthesis system 10 can accommodate a wide population of patients.

Cap 130 (or drive member) is internally threaded for engagement with the external threads 118 of proximal portion 111b of main body 110, as shown in FIG. 3B. Thus, rotating cap 130 moves cap 130 in a proximal-distal direction relative to main body 110. As mentioned above, alternative connection mechanisms may be implemented, such as a ratchet mechanism in which cap 130 may have teeth for engaging corresponding teeth of main body 110. Cap 130 also includes a distal face 136 that extends radially inwardly and is located at a proximal end of cap 130. Distal face 136 is generally planar and is configured to abut bushing 140, as described further below. Cap 130 defines an opening 132 which extends through distal face 136 and is configured to be coaxial with channel 104 of main body 110 when connected thereto.

Bushing 140 may be made from a biocompatible compliant material. Bushing 140 has an inner surface 142 and an outer surface 144 disposed opposite inner surface 142. Outer surface 144 may be a tapered surface which may taper inwardly in a distal direction and may be correspondingly tapered with inner surface 119 of proximal portion 111b of main body 110. Inner surface 142 may be cylindrical so as to match a corresponding cylindrical shape of adapter 52 of stem component 50. Thus, in embodiments in which adapter 52 is differently shaped, such as conical, for example, inner surface 142 of bushing 140 may be correspondingly shaped (e.g., conical). Bushing 140 may be a split ring or split annulus which may define two opposing walls 148, as shown in FIG. 3B. Such walls 148 may define a gap therebetween. When bushing 140 is received within channel 104 of main body 110 and driven in a distal direction, such as via cap 130, the tapered outer surface 144 of bushing 140 interacts with the tapered inner surface 119 of proximal portion 111b of main body 110, which contracts bushing 140 such that its inner cross-sectional dimension decreases, walls 148 move closer together, and the gap narrows. Conversely, where bushing 140 is moved proximally, bushing 140 may expand under its own bias such that walls 148 move farther apart and the gap between them widens. Bushing 140 may also have a circumferential flange 146 extending about a proximal end thereof which may abut a proximal end of proximal portion 111b of main body 110 to limit the distal travel of bushing 140 within main body 110, as depicted in FIG. 3B.

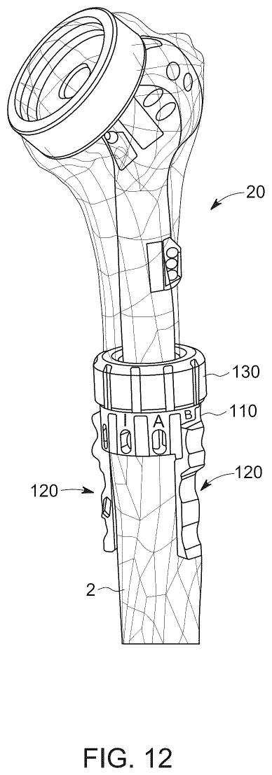

In an assembled condition, as shown in FIGS. 3A, 3B, and 12, three or more legs 120 may be connected to main body 110 and extend distally therefrom. Bushing 140 is positioned within proximal portion 111b of main body 110, and cap 130 is positioned over proximal portion 111b. When stem component 50 of endoprosthesis 20 is received within channel 114, flange 54 abuts shoulder 115 of main body 110, a distal end of bushing 140 is positioned proximal to flange 54, and bushing 140 extends about adapter 52 of stem component 50. Additionally, stem 51 of stem component 50 extends through distal portion 111a of main body 110 such that legs 120 are adjacent to and offset from stem 51. Thus, when stem component 50 is implanted into an intramedullary canal within a bone 2, the bone 2 is positioned between stem 51 and legs 120, and main body 110 rests on a resected surface of bone 2, as illustrated in FIG. 12. Stem component 50 may be further secured to bone clamp 100 by driving bushing 140 in a distal direction via cap 130. Thus, when cap 130 is rotated in a first rotational direction, end face 136 of cap 130 pushes bushing 140 in a distal direction thereby contracting bushing 140 onto adapter 52 of stem component 50. Bushing 140 may also abut against flange 54 of stem component 50 and squeeze flange 54 against shoulder 115. Thus, clamp 100 provides a snug and stable arrangement that distributes load both internally and externally to bone 2.

In addition to the descriptions above and the illustrations in the figures, various other operations are described below. These operations need not be performed in the exact order described. Steps may be performed in a different order, simultaneously, omitted, or added, unless otherwise specified.

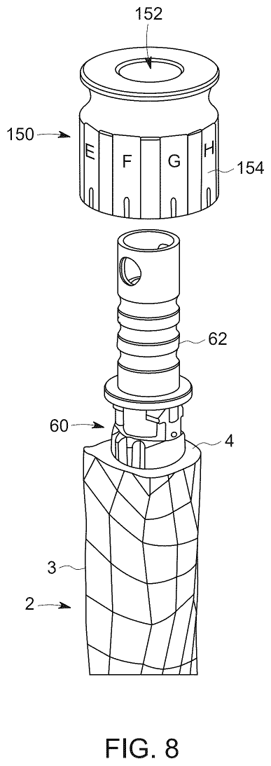

In an arthroplasty procedure, a large segment of bone may need to be removed due to severe trauma, bone tumors, or the like. In such a procedure, the bone 2 may be resected through a diaphysis of the bone to expose a resected surface. For example, in a proximal humeral reconstruction procedure, as shown in FIG. 8, the proximal humerus may be resected by cutting through the humeral shaft 3 of bone 2 to form a proximal resected surface 4. The intramedullary canal may be reamed or broached to shape it for receipt of intramedullary stem 50.

Prior to inserting intramedullary stem 50, a trialing procedure may be performed. In this regard, a trial stem 60 may be inserted into the intramedullary canal such that a proximal end 62 of stem 60 extends proximally therefrom, as shown in FIG. 8. For example, proximal end 62 of stem 60 may be an adapter. A main body sizer 150 may be placed over proximal end 62 of trial stem 60, such that proximal end 62 is received within a channel 152 of sizer 150. As shown, main body sizer 150 may have a plurality of facets 154 arrayed about its perimeter which may match the facet arrangement of main body 110 of bone clamp 100 and may have a size and shape corresponding to that of main body 110. The size of main body sizer 150 may be evaluated relative to bone 2, and if desirable, another main body sizer 150 of a different size may be exchanged until the appropriate size is achieved.

With the main body sizer 150 positioned on the resected surface 4 of bone 2 and disposed about the proximal end 62 of trial stem 60, an offset tool may then be utilized to assess the offset of bone 2 relative to the main body sizer 150 at various locations about bone 2 and main body sizer 150 for selection of appropriate legs 120 to be placed at the various locations.

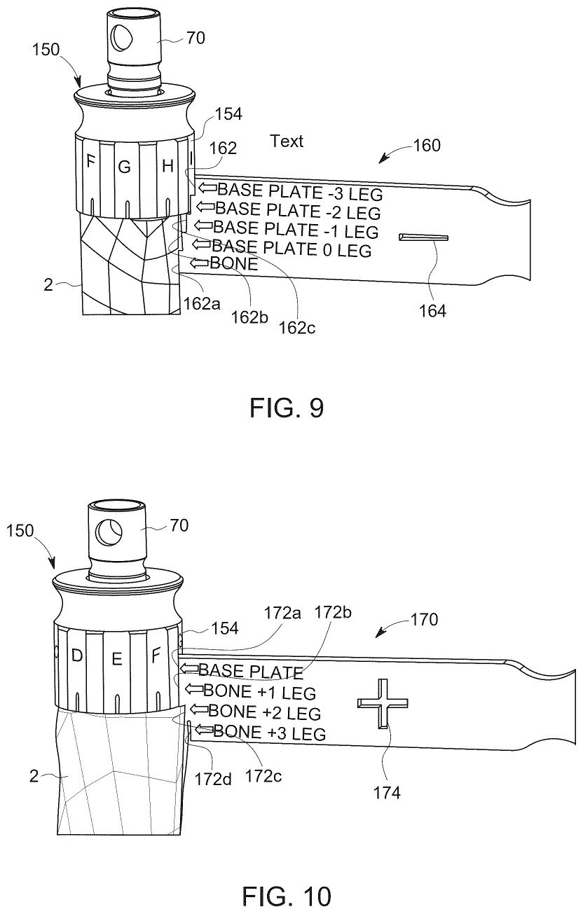

As shown in FIG. 9, a first offset tool 160 is configured to assess an inward offset of bone 2 relative to main body sizer 154. In this regard, first offset tool 160 includes a plurality of stepped surfaces 162 at a distal end thereof. For example, a first stepped surface 162a is a bone contact surface, a second stepped surface 162b is configured for contact with the main body sizer 150 and corresponds to a neutral offset, a third stepped surface 162c is configured for contact with the main body sizer 150 and corresponds to a first negative offset, and so on. Offset tool 160 may be provided with an indicator 164 to indicate that tool 160 is a negative offset (i.e., inward offset) tool.

FIG. 10 illustrates a second offset tool 170 which is configured for a positive offset (i.e., outward offset) of the bone. In this regard, second offset tool 170 is utilized where bone 2 projects outwardly from main body sizer 150, as opposed to first offset tool 160 which may be utilized when bone 2 is inwardly offset or flush with main body sizer 150. Thus, second offset tool 170 is similarly configured to first offset tool 160, but with the stepped surfaces 172 being stepped in an opposite direction than first offset tool 160. Additionally, with respect to second offset tool 170, first stepped surface 172a is configured to engage a facet 154 of main body sizer 150, while second, third, and fourth stepped surfaces 172b-d are configured to engage bone 2, for example.

Thus, as shown in FIGS. 9 and 10, offset determination may be performed by selecting a location about main body sizer 150 for measurement. Such location may be based on the location of a particular facet 154. In the case of an inward bone offset, first offset tool 160 is selected, and first stepped surface 162a is placed against bone 2, and another one of stepped surfaces 162 is placed against facet 154 of main body sizer 150. Whichever stepped surface 162 is able to contact main body sizer 150 while first stepped surface 162a remains in contact with bone 2 determines the leg offset for that particular location. When legs 120 are assembled to main body 110 of bone clamp 100, the selected leg 120 based on the measured offset is connected to opening 112 and corresponding facet 114 utilized during measurement. In other words, if a facet 154 labeled with a unique identifier of “A,” for example, on main body sizer 150 is used to measure a leg offset, leg 120 will be connected to an opening 112 corresponding to a facet 114 with the “A” identifier on main body 110 of bone clamp 100. Offset measurement with second offset tool 170 is similarly performed. Measurements are preferably taken at at least three locations about the perimeter of main body sizer 150.

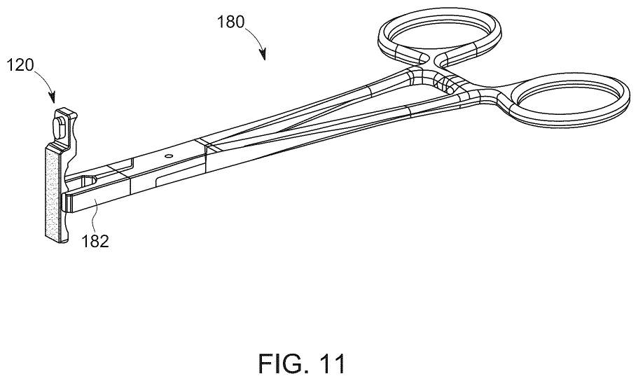

Once the appropriate leg offsets are determined, the selected legs 120 may be connected to main body 110. A grasper 180 may be used to facilitate the connections. Grasper 180 may be in the configuration of forceps, for example, which have arms 182 configured to engage tool engagement features 124 of legs 120 for secured handling, as shown in FIG. 11. Grasper 180 may then be used to connect each leg 120 to an appropriate opening 112, as described above.

Endoprosthesis 20 may then be inserted into bone clamp 100. However, it should be understood that legs 120 may be connected to main body 110 either before or after endoprosthesis 20 is connected to bone clamp 100. Endoprosthesis 20 is inserted through channel 104 of bone clamp 100 until flange 54 of stem component 50 rests against shoulder 115, and stem 51 extends distally from main body 110. The stem 51 of stem component 50 may be inserted into the intramedullary canal of bone 2 until distal surface 117 of main body 110 is positioned against resected surface 4. Bone clamp 100 may be rotated about stem component 50 to arrange legs 120 at the proper orientation along the cortical outer surface of bone 2 which may be performed before or after implantation of stem component 50. Cap 130 may be rotated to drive bushing 140 distally to squeeze adapter 52 of stem component 50 with bushing 140 and secure bone clamp 100 to endoprosthesis 20, as shown in FIG. 12. Thus, when endoprosthesis 20 is implanted into bone 2, stem 51 secures endoprosthesis 20 from within bone 2, and legs 120 engage and secure endoprosthesis 20 from outside of bone 2.

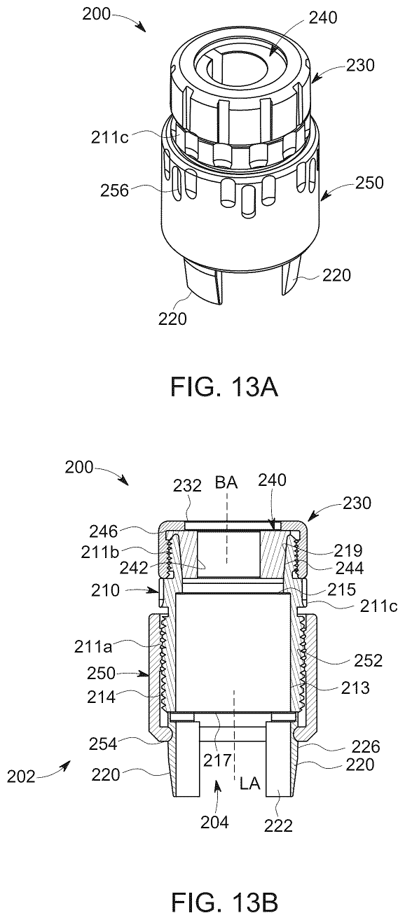

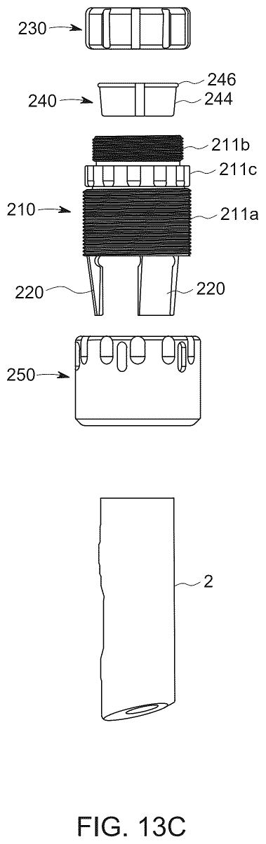

FIG. 13A-13C also depict a bone clamp 200 according to a further embodiment of the present disclosure. Bone clamp 200 is similar to bone clamp 200 except for the differences explicitly described and/or shown. Accordingly, similar elements are given corresponding reference numerals in the 200-series. For example, collet 202 corresponds to collet 102, cap 230 corresponds to cap 130, bushing 240 corresponds to bushing 140, and so on, except for the differences explicitly described or shown. In this regard, bone clamp 200, unlike bone claim 100, also includes a collar 250.

Collet 202 generally includes a main body 210 and a plurality of legs 220. In the embodiment depicted, legs 220 are integral with main body 210 so as to form a monolithic structure. Additionally, collet 202 is a moving-leg collet in that legs 220 are moveable radially outwardly and inwardly to engage bone, as described in more detail below.

Main body 210 may have a cylindrical exterior shape and cylindrical interior shape and has a longitudinal channel 204 extending therethrough which defines a longitudinal axis LA of main body 210 and of bone clamp 200. Distal portion 211a includes a distal surface 217 which is configured to engage a resected surface of a bone when implanted. Distal surface 217 may be correspondingly planar so as lay flush against the resected surface when implanted. Distal surface 217 may also have a porous structure to facilitate bone ongrowth or ingrowth. As shown, distal portion 211a includes external threading 214 for corresponding threaded engagement with collar 250. However, in other embodiments, distal portion 211a may have alternative features for connecting to collar 250, such as teeth of a ratchet mechanism, for example.

Proximal portion 211b of main body 210 defines a proximal end of collet 202. As shown, proximal portion 211b includes external threading 211b for corresponding threaded engagement with cap 230. However, in other embodiments, proximal portion 211b may have alternative features for connecting to cap 230, such as teeth of a ratchet mechanism, for example. Proximal portion 211b also includes an inner surface 219 which may be a tapered surface that tapers inwardly in a distal direction toward distal portion 211a. An annular shoulder 215 (or rim) may be formed on an interior of main body 210 and may be formed at a junction between proximal portion 211b and intermediate portion 211c. Such annular shoulder 215 is configured to receive flange 54 of stem component 50 such that flange 54 may abut shoulder 215 when stem component 50 is received within channel 204 of main body 210.

Intermediate portion 211c is disposed between distal portion 211a and proximal portion 211b. In the embodiment depicted, intermediate portion 211c has a cross-sectional dimension that is greater than a cross-sectional dimension of proximal portion 211b which creates a distal stop for cap 230, as illustrated in FIG. 13B.

Legs 220 extend distally from main body 210. As mentioned above, legs 220 are integrally connected to main body 210, such as to distal surface 217 of main body 210. Legs 220 are also cantilevered to main body 210 and are flexible such that they can be moved radially inwardly and radially outwardly. Legs 220 may also be biased inwardly or outwardly. Legs 220 have an inner surface 222 and an outer surface 226. Inner surface 222 of each leg 220 may be concavely curved. However, in some embodiments, inner surface 222 of each leg 220 may be flat. Inner surface 222 may also have a porous structure, or may otherwise be smooth, roughened, have corrugations, or have spikes, for example. Outer surface 226 of each leg 220 may be tapered so as to interact with collar to drive legs 220 radially inwardly, as discussed further below. In the embodiment depicted, collet 202 includes three legs 220. However, in other embodiments, more than three legs 220 may be provided.

Collar 250 has a cylindrical exterior shape and a cylindrical interior shape. However, in some embodiments, collar 250 may have a differing exterior shape, such as rectangular, hexagonal, octagonal, and the like. Collar 250 includes internal threads 252 which are configured to threadedly engage external threads 214 of distal portion 211a of main body 210. Collar 250 also includes a cam feature 254 (or rim) at a distal end thereof. Cam feature 254 may extend circumferentially about a longitudinal axis of collar 250. In operation, collar 250 may be driven distally relative to main body 210 which engages cam feature 254 with outer surfaces 226 of legs 220 and drives legs 220 radially inwardly toward longitudinal axis LA of main body 210. In this regard, when clamp 200 is positioned over a bone, collar 250 facilitates leg engagement with the bone and also resists their outward deflection.

Cap 230 and bushing 240 are similar to cap 130 and bushing 140. However, unlike bushing 140, bushing 240 may have an eccentric configuration. In this regard, bushing 240 may define a bushing axis BA which, when positioned within main body 210, may be offset relative to longitudinal axis LA of main body 210, as shown in FIG. 13B. This eccentricity can account for offsets of the stem component 50 relative to the bone. Thus, bone clamp 200 may be provided with a kit that includes a concentric bushing 240 and multiple bushings 240 with various eccentricities. For example, a kit may include a first and second eccentric bushings 240 with a 1 mm and 2 mm offsets, respectively, and a third eccentric bushing 240 with a 0 mm offset (i.e., a concentric bushing). Although bushing 140 of bone clamp 100 may generally be of the concentric type as the modular legs 120 thereof can account for stem-bone offsets, it is also contemplated that an eccentric bushing, like bushing 240, may be provided therewith.

In an assembled condition, bushing 250 is positioned within proximal portion 211b of main body 210, cap 230 is positioned over proximal portion 211b, and collar 250 is disposed over distal portion 211a of main body 210. When stem component 50 of endoprosthesis 20 is received within channel 204, flange 54 abuts shoulder 215 of main body 210, a distal end of bushing 240 is positioned proximal to flange 54, and bushing 240 extends about adapter 52 of stem component 50. Additionally, stem 51 of stem component 50 extends through distal portion 211b of main body 210 such that legs 220 are adjacent to and offset from stem 251. Thus, when stem component 50 is implanted into an intramedullary canal within a bone, the bone is positioned between stem 51 and legs 220, and distal surface 217 of main body 210 rests on a resected surface of the bone. Collar 250 may then be rotated in a first direction which drives collar 250 in a distal direction. As collar 250 is driven in a distal direction, cam feature 254 contacts legs 220 and drives them inwardly to securely engage them with the bone. Stem component 50 may be further secured to bone clamp 200 by driving bushing 240 in a distal direction via cap 230, as described above with respect to clamp 100.

FIG. 14A depicts a bone clamp 300 according to another embodiment of the present disclosure. Bone clamp 300 is similar to bone clamp 200 in that it includes a collet 302 with a main body 310 and integral legs 320 connected to main body 310. Additionally, bone clamp 300 includes a bushing 340, which may be an eccentric bushing or concentric bushing, and a cap 330 for driving bushing 340 into engagement with an intramedullary stem, such as stem component 50. However, unlike bone clamp 200, bone clamp 300 may not include a collar for actuating legs 320 inwardly. Instead, legs 320 may be biased radially inwardly and, when positioned over a cortical outer surface of a bone, legs 320 may flex outwardly while applying inward radial force against the cortical outer surface. As shown, bone clamp 300 may include a plurality of legs 320, such as more than three legs 320. For example, in the embodiment depicted, bone clamp 300 includes six legs 320.

FIG. 14B depicts an exemplary kit of bone clamps like that of bone clamp 300. Such kit may include a first bone clamp 300a, a second bone clamp 300b, and a third bone clamp 300c each of different size. For example, first bone clamp 300a may have a nominal diameter size of 27 mm, second bone clamp 300b may have a nominal diameter size of 24 mm, and third bone clamp 300c may have a nominal diameter size of 21 mm. Additionally, kit may include a plurality of bushings 340 which can be modularly received in any of bone clamps 300a-b. For example, the kit may include a first and second eccentric bushings 340a, 340b with a 1 mm and 2 mm offset, respectively, and a third eccentric bushing 340c with a 0 mm offset.

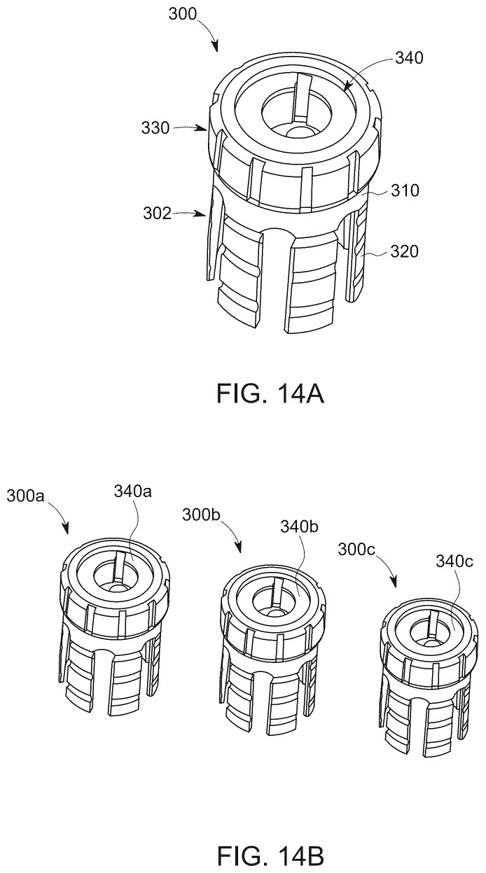

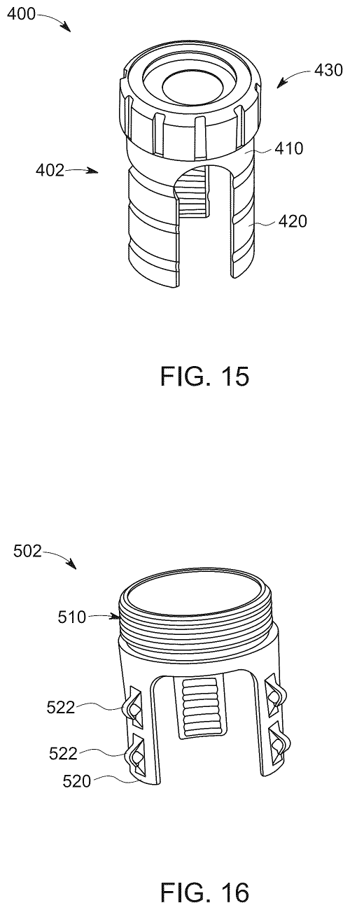

FIG. 15 also depicts a bone clamp 400 according to a further embodiment of the present disclosure. Bone clamp 400 is similar to bone clamp 300 except for the differences explicitly described and/or shown. Accordingly, similar elements are given corresponding reference numerals in the 400-series. For example, cap 430 corresponds to cap 330, bushing 440 corresponds to bushing 340, and so on, except for the differences explicitly described or shown. In this regard, bone clamp 400 differs in that collet 402 includes three legs 420. Legs 420 may curve about a longitudinal axis of collet 402 and may have an arc length defined about the longitudinal axis. As illustrated, the arc lengths of each leg 420 may be greater than that for legs 320 of bone clamp 300. This greater arc length can increase the bending resistance as compared to legs 320 to account for the reduced number of legs 420 relative to that of bone clamp 400.

FIG. 16 depicts a collet 502 according to another embodiment of the present disclosure. Collet 502 is similar to collet 402 in that it has a plurality of legs 420 integrally connected to main body 510 and, therefore, may be utilized in bone clamp 400 in lieu of collet 402, for example. However, unlike collet 402, each leg 420 includes one or more eyelets 522 disposed on an exterior thereof. For example, each leg 520 may include two eyelets 522 offset from each other in an axial direction. Eyelets 522 are each configured to receive a suture or wire (e.g., cerclage wire). This allows a suture or wire to be threaded through eyelets 522 of each leg 520 to secure legs 520 against the cortical outer surface of a bone so as to increase clamping force and resist radial outward deflection.

Although the aforementioned exemplary embodiments have been described with respect to exemplary proximal humeral endoprosthesis 20 and in the context of proximal humeral limb salvage, it should be understood that such embodiments may also be applied to other long bones and endoprostheses therefor. For example, the aforementioned cortical bone clamps 100, 200, 300, 400 may be implemented in the context of distal humeral limb salvage, femoral limb salvage (proximal and distal femur), and tibial limb salvage (proximal and distal tibia), for example. As such, the herein described cortical bone clamps 100, 200, 300, 400 may be implemented in conjunction with distal humeral endoprostheses, proximal femoral endoprostheses, distal femoral endoprostheses, proximal tibial endoprostheses, and distal tibial endoprostheses, for example. In addition, some limb salvage procedures may remove a middle segment of a long bone such that the end portions of the long bone that include native articular surfaces may be spared. An implant replacing the middle segment of bone, referred to herein as an intercalary prosthesis, may utilize one or more of the aforementioned cortical bone clamps 100, 200, 300, 400. An exemplary intercalary prosthesis is described in U.S. Publication No. 2021/0378827, the disclosure of which is incorporated herein by reference in its entirety.

Although the subject matter disclosed herein has been described with reference to specific embodiments, these are merely illustrative of the principles and applications discussed. Numerous modifications can be made to these embodiments, including combining features from different embodiments. Therefore, the exemplary embodiments are not intended to be exhaustive or to limit the disclosed subject matter.

1. A bone clamp for coupling an intramedullary stem to a bone in which the intramedullary stem is received, the bone clamp comprising:
a collet having a main body defining a proximal end thereof and a plurality of legs extending distally from the main body, the collet forming an internal channel configured to receive at least a portion of the bone and at least a portion of the intramedullary stem therethrough;
a collar having internal threading that is complementary to external threading on the main body of the collet, the collar being configured to be received around the main body of the collet such that rotation of the collar relative to the collet translates the collar distally relative to the collet to compress the plurality of legs radially inwardly;
a bushing configured to be received within the proximal end of the collet, the bushing having a longitudinal channel configured to receive at least a portion of the intramedullary stem therethrough, the bushing being generally annular with a gap formed between two walls of the bushing that confront each other; and
a cap having internal threading that is complementary to external threading on the proximal end of the collet, the cap having a longitudinal channel configured to receive at least a portion of the intramedullary stem therethrough, the cap having a distal face configured to contact a proximal face of the bushing, the cap being configured to be received around the proximal end of the collet such that such that rotation of the cap relative to the collet translates the cap distally relative to the collet to compress the bushing thereby reducing a size of the gap and compressing the bushing onto the intramedullary stem.

2. The bone clamp of claim 1, wherein the legs of the collet are integrally formed with the main body so as to form a monolithic structure.

3. The bone clamp of claim 2, wherein the legs are cantilevered to the main body and are deflectable from a first position to a second position, the legs being biased towards the first position.

4. The bone clamp of claim 3, wherein the legs each have an exterior surface, and the collar includes a cam surface engaging the exterior surface of each of the legs such that driving the collar in a distal direction cams the legs towards the second position.

5. The bone clamp of claim 1, wherein the main body includes a proximal end portion defining the proximal end of the collet, the proximal end portion having the external threading complementary to the internal threading of the cap.

6. The bone clamp of claim 5, wherein the main body includes a distal end portion defining a distal end of the collet, the distal end portion having the external threading complementary to the internal threading of the collar.

7. The bone clamp of claim 5, wherein the proximal end portion of the main body includes a tapered inner surface, and the bushing is received within the inner surface and includes a tapered outer surface complementary to the tapered inner surface of the proximal end portion such that driving the bushing in a distal direction closes the gap between the two walls of the bushing.

8. The bone clamp of claim 1, wherein the collar includes tool engagement features on an outer surface thereof for engagement with a torque applying tool.

9. The bone clamp of claim 1, wherein the main body includes a circumferential shoulder at an interior thereof for receipt of a corresponding flange of the intramedullary stem.

10. The bone clamp of claim 9, wherein the circumferential shoulder is disposed distally to the bushing, such that driving the bushing distally when the flange of the intramedullary stem abuts the circumferential shoulder, the bushing engages the flange.

11. The bone clamp of claim 1, wherein the bushing is eccentric such that a longitudinal axis of the bushing defined by the longitudinal channel of the bushing is offset relative to a longitudinal axis of the collet defined by the channel of the collet.

12. The bone clamp of claim 1, wherein the bushing is made from a compliant material.

13. The bone clamp of claim 1, wherein the bushing includes a circumferential flange disposed between the proximal end of the collet and the cap so as to constrain distal movement of the bushing.

14. The bone clamp of claim 1, wherein the plurality of legs of the collet includes at least three legs.

15. A bone clamp for coupling an intramedullary stem to a bone in which the intramedullary stem is received, the bone clamp comprising:
a collet comprising:
a main body defining a proximal end portion defining a proximal end of the collet, the main body forming an internal channel configured to receive at least a portion of the bone and at least a portion of the intramedullary stem therethrough, the main body having a plurality of openings formed in a side wall thereof, the openings being spaced apart from each other in a circumferential direction of the main body, and
a plurality of legs, each of the plurality of legs having a distal portion having an inner surface configured to contact the bone, and a proximal portion having a protrusion, each protrusion of the plurality of legs being configured to be received within any one of the openings formed in the side wall of the main body to couple corresponding leg to the main body;
a bushing configured to be received within the proximal end portion of the main body, the bushing having a longitudinal channel configured to receive at least a portion of the intramedullary stem therethrough, the bushing being generally annular with a gap formed between two walls of the bushing that confront each other; and
a cap having internal threading that is complementary to external threading on the proximal end portion of the main body, the cap having a longitudinal channel configured to receive at least a portion of the intramedullary stem therethrough, the cap having a distal face configured to contact a proximal face of the bushing, the cap being configured to be received around the proximal end portion of the main body such that such that rotation of the cap relative to the main body translates the cap distally relative to the main body to compress the bushing to reduce a size of the gap of the bushing.

16. The bone clamp of claim 15, wherein the inner surface of each of the plurality of legs includes a porous structure.

17. The bone clamp of claim 16, wherein the inner surface of each of the plurality of legs is flat.

18. The bone clamp of claim 15, wherein the proximal portion of each of the legs defines a first longitudinal axis, and the distal portion of each of the legs defines a second longitudinal axis.

19. The bone clamp of claim 18, wherein the plurality of legs includes a first leg, and the first longitudinal axis of the distal portion of the first leg is offset relative to the second longitudinal axis of the distal portion of the first leg.

20. The bone clamp of claim 19, wherein the second longitudinal axis of the distal portion of the first leg is offset radially inwardly relative to the first longitudinal axis of the proximal portion of the first leg when the first leg is connected to the main body.
